(19) United States
(12) Patent Application Publication
(10) Pub. No.: US 2016/0096797 A1
(43) Pub. Date: Apr. 7, 2016

(54) METHOD OF SEPARATING LACTIC ACID OR SALT THEREOF

(71) Applicant: Samsung Electronics Co., Ltd., Suwon-si (KR)

(72) Inventors: Jaeeun Kim, Hwaseong-si (KR); Donwook Lee, Seoul (KR); Jinkyu Kang, Hwaseong-si (KR); Eunsuk Kwon, Suwon-si (KR); Hyerim Gwon, Suwon-si (KR); Kwangmyung Cho, Seongnam-si (KR)

(21) Appl. No.: 14/877,719

(22) Filed: Oct. 7, 2015

(30) Foreign Application Priority Data
Oct. 7, 2014 (KR) 10-2014-0135123

(51) Int. Cl. C07C 51/42; C12P 7/56; B01D 69/02; B01D 15/08; B01D 15/36; C07C 51/47; B01D 61/02

(52) U.S. Cl. CPC C07C 51/42; C07C 51/47; C12P 7/56; B01D 61/027; B01D 15/08; B01D 15/361; B01D 69/02; B01D 2311/2623; B01D 2311/2626; B01D 2325/20

(57) ABSTRACT

Provided is a method of separating lactic acid or a salt thereof. The method includes removing a coloring material, including hydroxymethylfurfural (HMF), from a fermentation broth, which includes at least one of lactic acid and a salt thereof; and recovering lactic acid or a salt thereof from the fermentation broth, from which the coloring material has been removed.

### RELATED APPLICATION

This application claims the benefit of Korean Patent Application No. 10-2014-0135123, filed on Oct. 7, 2014, in the Korean Intellectual Property Office, the entire disclosure of which is hereby incorporated by reference.

### BACKGROUND

1. Field

The present disclosure relates to methods of separating lactic acid or a salt thereof.

2. Description of the Related Art

Lactic acid is being widely used in a variety of industries and fields such as the food industry and the pharmaceutical, chemical, and electronic fields. Lactic acid is a colorless, odorless, and low-volatility material that is easily soluble in water. Lactic acid that is not toxic to the human body is used as a flavoring agent, an acidifier, or a preserving agent. Also, lactic acid is a raw material of biodegradable polylactic acid (PLA) that is an alternative environmentally friendly polymeric material. Technically, PLA is polyester-based resin that is obtained by conversion of PLA into lactide in the form of a dimer and then by ring-opening polymerization for polymerization. In addition, PLA is available in various processing forms such as films, sheets, fiber, or injections. In order to produce lactic acid that is a raw material of PLA, a purification process that allows obtaining high-purity lactic acid by completely removing impurities from lactic acid is required. In order to obtain high-purity lactic acid, it is essential to produce optically transparent lactic acid even at a high temperature.

In the modern industry, lactic acid is produced by petrochemical synthesis and biotechnological fermentation. The lactic acid produced by biotechnological fermentation may be purified by precipitation, extraction, adsorption, reactive distillation, electro dialysis, or nano filtration membrane. Generally, a method of purifying lactic acid may be, for example, carried out by precipitating calcium sulfate after acidification, adsorbing the lactic acid on an ion exchange resin by passing through an activated carbon substrate, and distilling the resultant material.

However, it is difficult to completely remove a coloring material such as hydroxymethylfurfural (HMF) only with the method of purifying lactic acid because the coloring material is present in the lactic acid in a trace amount even at a high temperature during the manufacture of PLA. Therefore, there is a need for a new purification method to obtain optically transparent high-purity lactic acid even at a high temperature.

### SUMMARY

Provided is a novel method of separating lactic acid or a salt thereof. The method comprises removing a coloring material comprising hydroxymethylfurfural (HMF) from a fermentation broth, wherein the fermentation broth comprises lactic acid or a salt thereof; and recovering the lactic acid or a salt thereof from the fermentation broth after the coloring material has been removed.

Additional aspects will be set forth in part in the description which follows and, in part, will be apparent from the description, or may be learned by practice of the presented exemplary embodiments.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a graph of residual hydroxymethylfurfural (HMF) concentration (mg/L) and chromaticity of recovered lactic acid as evaluated in Examples 1 to 3 and Comparative Examples 1 and 2;

FIG. 2 is a flowchart of a method of purifying lactic acid, according to an embodiment; and

FIG. 3 is a flowchart of a method of purifying lactic acid, according to another embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Provided herein is a method of separating lactic acid or a salt thereof from a fermentation broth. The method comprises removing a coloring material, including hydroxymethylfurfural (HMF), from the fermentation broth, which may include at least one of lactic acid and a salt thereof; and recovering lactic acid or a salt thereof from the fermentation broth, from which the coloring material has been removed.

The method of separating lactic acid and a salt thereof includes removing a coloring material including HMF, thereby recovering optically transparent lactic acid even at a high temperature.

In some embodiments, lactic acid and a salt thereof may be separated as follows:

A fermentation broth including at least one of lactic acid and a salt thereof may be prepared by any suitable method.

The fermentation broth including the lactic acid or salt thereof may be prepared, for example, via biotechnological fermentation. The biotechnological fermentation is carried out by using a renewable carbohydrate such as starch, sucrose, maltose, glucose, fructose, and xylose, as a substrate to culture and ferment a microorganism. Lactic acid or a salt thereof may be produced more effectively when using biotechnological fermentation than petrochemical synthesis that involves several steps of chemical reactions.

The microorganism used in the fermentation broth may be a lactic acid-producing microorganism. Any microorganism may be used as long as the microorganism is capable of producing lactic acid. The lactic acid-producing microorganism may be, for example, at least one selected from Bacillus, Lactobacillus, Lactococcus, and Streptococcus. Alternatively, the lactic acid-producing microorganism may be at least one selected from Candida, Saccharomyces, Shizosaccharomyces, Kluyveromyces, Pichia, Issachenkia, and Hansenula. For example, the lactic acid-producing microorganism may be Kluyveromyces marxianus, Saccharomyces cerevisiae, or Ceratomia sonorensis.

Examples of a fermentation process include a batch fermentation process, a continuous fermentation process, or a fed-batch fermentation process. However, the method claimed herein is not limited thereto, and any fermentation processes available in the art may be used. A culture medium may be obtained, for example, from a mixture prepared by adequately mixing at least a lactic acid-producing microorganism, pentose (C₅ sugar), hexose (C₆ sugar), starch, and cellulose such as lignocellulose. During the fermentation process, the pH of a pre-fermentation broth may change from neutral pH to acidic pH. The fermentation broth may be overly acidified at a pH in a range of about 2.0 to about 3.5. If the lactic acid-producing microorganism, which was used in the culture medium, does not endure the overly acidified environment, a basic compound may be added to the fermentation broth in order to provide an environment of which the pH is approximately neutral.

The basic compound is capable of adjusting the pH of the fermentation broth in a range of about 3.5 to about 6. The basic compound may include at least one of NaOH, Ca(OH)₂, Mg(OH)₂, KOH, Ba(OH)₂, and NH₄(OH). For example, the basic compound may be NH₄(OH) or Ca(OH)₂. The pre-fermentation broth to which the basic compound is added may be fermented so that a lactate concentration of the broth may increase by forming, for example, ammonium lactate (NH₄(LA)) or calcium lactate (Ca(LA)₂). Lactic acid or a salt thereof may be easily separated from an inorganic material in the fermentation broth by adding acid after fermentation, in order to prepare a fermentation broth having a high yield of lactic acid or a salt thereof.

Cells may be removed from the prepared fermentation broth. The term “cell” is used herein in a broad sense including “bacteria” or “yeast strain”. For example, the “cell” may be a “yeast strain”.

The method of removing the cells is not particularly limited, and may include using a centrifuge, a filter press, a diatomite filter, a rotary vacuum filter, a membrane filter, or aggregation and suspension.

For example, a centrifuge may be used to remove the cells. The centrifuge provides a method of separating a fermentation broth, including a cell and at least one of lactic acid or a salt thereof, by utilizing density differences. For example, cells may be easily removed from the fermentation broth including at least one of lactic acid and a salt thereof by centrifuging the fermentation broth at a rotation speed in a range of about 1000 to about 4000 rpm for about 1 to about 20 minutes. Examples of apparatuses used in centrifugation are a centrifugal settler and a centrifugal filter. The method of using a centrifuge may be performed with other filtration methods at the same time.

Preparing the fermentation broth may further include concentrating the fermentation broth. The fermentation broth may be concentrated by removing ingredients that are volatile, such as ethanol or water from the fermentation broth. In order to remove the ingredients including ethanol or water, a vacuum evaporation method and/or an evaporative concentration method may be performed by using a vacuum evaporator or a rotary vacuum evaporator. A fermentation broth, from which the cell has been removed, for example, may be concentrated under a condition of a low temperature and reduced pressure. For example, the concentrating the fermentation broth may be performed at a temperature in a range of about 40° C. to about 80° C. and under a pressure in a range of about 10 torr to about 70 torr for about 1 hour to about 5 hours. A volume of the concentrated fermentation broth may be in a range of about 40 volume % to about 70 volume % of a volume of the starting fermentation broth, from which the cells have been removed. In some embodiments, the volume of the concentrated fermentation broth may be about 50 volume % to about 70 volume %.

Preparing the fermentation broth may further include removing an inorganic material by adding an acid into the fermentation broth. The acid may include, for example, sulfuric acid, hydrochloric acid, nitric acid, hydrobromic acid (HBr), hydriodic acid (HI), phosphoric acid (H₃PO₄), and/or perchloric acid (HClO₄). For example, the acid may be sulfuric acid since sulfuric acid has low water concentration and is easily handled. When adding sulfuric acid into the fermentation broth, an inorganic material such as calcium sulfate (Ca(SO₄)₂) including calcium ions (Ca²⁺) present in the fermentation broth may be precipitated. Addition of sulfuric acid may form lactic acid from a basic-cation lactate such as ammonium lactate (NH₄(LA)) or calcium lactate (Ca(LA)₂) produced from the addition of a basic compound such as NH₄(OH) or Ca(OH)₂ added for adjusting pH. The sulfuric acid may be added up to an amount at which the inorganic material precipitates. For example, an additive amount of the sulfuric acid may be in a range of about 0.1- to about 3-fold molar ratio with respect to lactic acid or a salt thereof included in the fermentation broth. Thereafter, the fermentation broth may be cooled to room temperature, and a precipitated inorganic material may be removed by a filtration process. By performing the filtration process using a known filter under a vacuum condition, high-purity lactic acid may be recovered.

A coloring material including HMF may be removed from the fermentation broth. The term “coloring material” as used herein includes, in a broad sense, levoglucosan, furfural, erythrose, glyceraldehydes, pyruvaldehyde, a coloring material known as melanodin, a coloring material having 500 Dalton of average molecular weight such as caramelans (C₂₄H₂₆O₁₈), caramelens (C₃₆H_{SO}O₂₅), caramelins (C₁₂₅H₁₈₈O₈₀), in addition to HMF. An organic material can comprise levoglucosan, furfural, erythrose, glyceraldehydes, pyruvaldehyde, and/or hydroxymethylfurfural (HMF), such as that formed by a Maillard reaction between an amino group of amino acids or peptides and a carbonyl group of reduced sugars; ingredients derived from a natural product such as yeast; or an inorganic ion. The organic material may also include HMF, amino acids, peptides, or sugars, for example.

The removing of the coloring material may include filtering the fermentation broth, such as by allowing the fermentation broth to pass through a nano-filter membrane. The term “allowing a fermentation broth to pass through a nano-filter membrane” used herein indicates removing of the coloring material by filtering the fermentation broth through a nano-filter membrane, which blocks the coloring material. The lactic acid or a salt thereof may be included in filtrate that passed through the nano-filter membrane.

The nano-filter membrane may have a molecular weight cutoff (MWCO) in a range of about 100 to about 800 Daltons, such as about 200 to about 600 Daltons, or about 200 to about 500 Daltons. A material of the nano-filter membrane may be a polyamide, for example, piperazine or cross-linked piperazine, but is not limited thereto. The material of the nano-filter membrane may also comprise celluloses, polyesters, or a polymer nano-filter membrane material such as a vinyl polymer. The nano-filter membrane may be a single membrane; however, in some cases, the nano-filter membrane may include a plurality of membranes.

The nano-filter membrane may have a plurality of nanometer-sized minute voids in a surface thereof, and may have a membrane structure in which there are minute voids branching in different directions. When the nano-filter membrane has an MWCO in the above-described range, organic material, including HMF, may be captured by a plurality of the minute voids in the surface thereof and/or in the membranes such that permeating of the organic material may be difficult, but lactic acid or a salt thereof may easily pass through the nano-filter membrane. Examples of a nano-filter membrane having an MWCO may include NF270 (available from Dow Filmtec, MWCO: 200 Da), TS40 (available from Trisep, MWCO: 300 Da), or XN45 (available from Trisep, MWCO: 500 Da) and the like. The HMF-removing ability of NF270 (available from Dow Filmtec, MWCO: 200 Da), TS40 (available from Trisep, MWCO: 300 Da), or XN45 (available from Trisep, MWCO: 500 Da) and the permeability of lactic acid or a salt thereof will be described below in this specification.

The filtering of the fermentation broth may include allowing the fermentation broth to pass through a filter after a material that adsorbs a coloring material has been used to batch-adsorb the coloring material. The material adsorbing a coloring material may include at least one selected from an activated carbon, a styrene polymer, and a cross-linked polystyrene polymer. For example, the material adsorbing the coloring material may be an activated carbon or an activated carbon powder.

An average pore diameter of the activated carbon may be in a range of about 2.3 nm to about 3.5 nm. A pore volume of the activated carbon measured by using a Barett-Joyner-Halenda (BJH) method may be in a range of about 0.20 cm³/g to about 0.60 cm³/g. For example, the pore volume may be in a range of about 0.20 cm³/g to about 0.55 cm³/g. For example, the pore volume may be in a range of about 0.20 cm³/g to about 0.52 cm³/g. Adsorbing and filtering the fermentation broth using an activated carbon having an average pore diameter in the above-described range and a pore volume by using a BJH method greatly improves the efficiency of HMF removal.

Examples of the coloring material may include HMF, amino acids, peptides, and sugars. Examples of the divalent inorganic ion may include Ca²⁺ and Mg⁺².

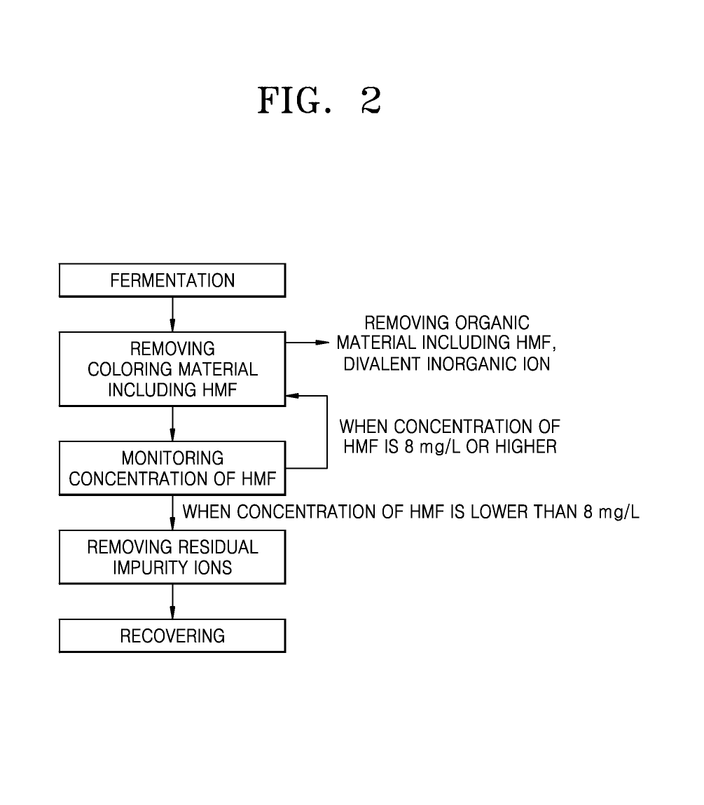

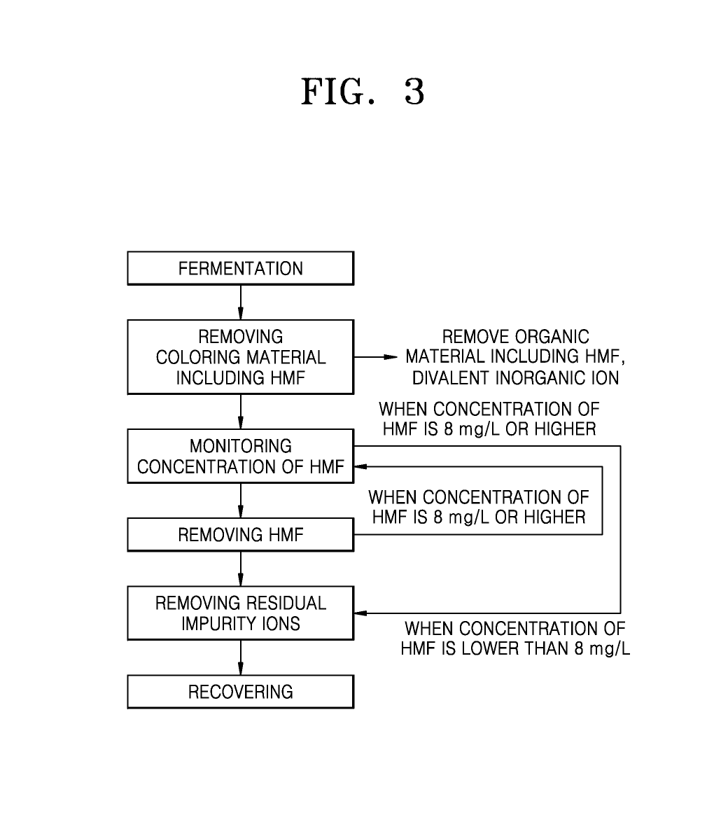

The method may further include monitoring a concentration of HMF included in the fermentation broth after the removing of the coloring material and/or determining whether to perform additional HMF removal. FIGS. 2 and 3 are flowcharts of methods of purifying lactic acid, according to embodiments of the method described herein.

As depicted in FIGS. 2 and 3, HMF concentration may be monitored after removing of the coloring material, including HMF, from the fermentation broth. Additional HMF removal may be appropriate when the HMF concentration is 8 mg/L or more. Any filtration method in the art may be used for the additional HMF removal. For instance, the method of removing a coloring material described herein can be repeated, or a different method of removing the coloring material as may be known in the art can be used. Subsequently, further monitoring a concentration of HMF may be performed. When the concentration of HMF in the fermentation broth is lower than 8 mg/L, the process may proceed to the next operation. The above described process may be performed once or repeated as necessary.

The monitoring of the concentration of HMF may be performed by using a liquid chromatography (LC) method or a high performance liquid chromatography (HPLC) method.

The method may further include removing residual impurity ions before the recovering lactic acid or a salt thereof. The term “removing residual impurity ions” as used herein includes “metal cations”. The metal cations may include, for example, Ca²⁺, Na²⁺, or Mg²⁺.

The removing of residual impurity ions may include allowing the fermentation broth, from which the coloring material has been removed, to pass through a nano-filter membrane, an ion exchange resin or an activated carbon. The removing of residual impurity ions also may be performed by using dialysis or electro dialysis.

When using an ion exchange resin, the fermentation broth may be sequentially contacted with a cation exchange resin and an anion exchange resin. For the cation exchange resin, a strong-acid cation exchange resin and/or a weak-acid cation exchange resin may be used. Since the strong-acid cation exchange resin is capable of exchanging cations in a wide pH range, the strong-acid cation exchange resin may be used as a cation exchange resin. For the anion exchange resin, a strong-base anion exchange resin and a weak-base anion exchange resin may be used. The strong-base anion exchange resin has excellent ion exchange capacity and adsorbability, and the weak-base anion exchange resin has excellent regenerability.

The sequentially contacting the ion exchange resin with the cation exchange resin and the anion exchange resin may include, for example, allowing contact with a strong-acid cation exchange resin and a weak-base anion exchange resin or allowing contact with a strong-acid cation exchange resin, a weak-base anion exchange resin, and a mixed ion exchange resin. For example, the sequentially contacting the ion exchange resin with the cation exchange resin and the anion exchange resin may include sequentially contacting with a strong-acid cation exchange resin, a weak-base anion exchange resin, a strong-acid cation exchange resin, and a strong-base anion exchange resin. The sequentially contacting with the cation exchange resin and the anion exchange resin may be performed once or repeated as necessary.

The recovering lactic acid may include distilling the fermentation broth, from which the coloring material has been removed.

The distilling may include thin film distillation or batch distillation The thin film distillation may include heating under a high vacuum condition and forming a film layer by using the fermentation broth on an inner wall of a distiller by using a wiper in order to increase thermal efficiency to a maximum and control heat contact time, and thus, a material, which is unstable to heat, may be effectively separated, compared to batch distillation. For example, the thin film distillation may be performed under a pressure in a range of about 1 torr to about 5 torr and at a temperature in a range of about 100° C. to about 170° C.

The recovering lactic acid may further include concentrating the fermentation broth, from which the coloring material has been removed, before the distilling of the fermentation broth, from which the coloring material has been removed.

The concentrating the fermentation broth, from which the coloring material has been removed, may be performed by using a vacuum evaporation method and/or an evaporative concentration method by using a vacuum evaporator or a rotary vacuum evaporator. For example, the fermentation broth, from which the coloring material has been removed, may be concentrated under a condition of a low temperature and reduced pressure. For example, concentrating the fermentation broth, from which the coloring material has been removed, may be performed at a temperature in a range of about 40° C. to about 80° C. and under a pressure in a range of about 1 torr to about 30 torr for about 1 hour to about 5 hours. A concentrated fermentation broth, from which the residual impurity ions have been removed, may have a water content of about 5 volume % or less.

A chromaticity of the recovered lactic acid is APHA color of about 50 or less when the lactic acid is heated to maintain a temperature of about 200° C. for about 2 hours and a concentration of the lactic acid is 90 wt %. Accordingly, the recovered lactic acid may have an optically transparent chromaticity even at a high temperature.

The inventive concept will be described in further detail with reference to the following examples and comparative examples. These examples are for illustrative purposes only and are not intended to limit the scope of the inventive concept.

### EXAMPLE

### Example 1

1.1 Preparing Fermentation Broth

In a 30 L fermenter containing a 20 L medium, a lactic acid-producing yeast strain, which is Saccharomyces cerevisiae (CEN.PK2-1C), was added, and fed-batch fermentation was performed thereon, to obtain a fermentation broth containing lactic acid. Medium ingredients include, for example, carbon sources, nitrogen sources, trace elements, vitamins, or amino acids, and their concentrations are as follows: glucose 80 g/L, yeast extract 10 g/L, KH₂PO₄ 2 g/L, (NH₄)₂SO₄ 2 g/L, MgSO₄ 1 g/L, leucine 0.1 g/L, Valine 0.05 g/L, Tryptophan 0.1 g/L, Histidine 0.05 g/L, Uracil 0.1 g/L, Inositol 0.1 g/L, FeSO₄.7H₂O 0.075 g/L, MnSO₄.5H₂O 0.02 g/L, CaCl₂.2H₂O 0.0025 g/L, CuSO₄.5H₂O 0.002 g/L, ZnSO₄.7H₂O 0.02 g/L, H₃BO₃ 0.002 g/L, and Na₂MnO₄ 0.002 g/L. Particularly, the fed-batch fermentation was performed by stirring at 200 rpm by using a single, midlevel, upflow marine-type impeller and by incubating at a temperature of 36° C. Ca(OH)₂ (5M) was automatically added to maintain the pH of the fermentation broth at 3.7.

Here, the fermentation broth containing lactic acid was collected after a lapse of about 48 hours from the start of the fermentation in the 30 L fermenter.

The yeast strain was removed from the fermentation broth containing lactic acid by centrifuging the fermentation broth containing lactic acid by using a centrifuge (Continent512R plus, available from Hanil science industrial, Max RCF: 6,359×g) for 15 minutes. 20 L of a fermentation broth, from which the yeast strain has been removed, was evaporated by using a rotary vacuum evaporator at a temperature of 70° C. and under a pressure of 50 torr for 2 hours. By doing so, ingredients including ethanol were removed therefrom, thereby obtaining a first concentrated fermentation broth, from which the yeast strain has been removed, that is concentrated to 50 volume % of the original volume.

98% sulfuric acid (1 eq.) was added to 10 L of the first concentrated fermentation broth until a pH of the fermentation broth decreased to pH 2.37, thereby causing precipitation of gypsum (Ca(SO₄)₂) and forming lactic acid from calcium lactate. The first concentrated fermentation broth was cooled to room temperature and was vacuum-filtered to obtain a prepared fermentation broth, from which precipitated gypsum (Ca(SO₄)₂) has been removed.

1.2 Removing Coloring Material Including HMF

8 L of the prepared fermentation broth was filtered by using a cross-flow filter apparatus (available from Cheon-ha Industry) having a nano-filter membrane NF270 membrane (available from Dow Filmtec) in order to remove a coloring material including organic material such as HMF or glucose or a divalent inorganic ion. Here, operating pressure was 40 bars. Here, the MWCO of the nano-filter NF270 membrane was 200 Daltons.

Subsequently, an activated carbon (DX) (available from Carbon-norit, 10 w/w lactic acid %) was added to the result, and the result was stirred at room temperature for 2 hours. Here, an average pore diameter of the activated carbon (DX) is 2.9 nm, the BET specific surface area is 1045 m²/g, and a pore volume measured by using a BJH method is 0.45 cm³/g. The stirred result was vacuum-filtered by using a disposable filter in order to remove carbon, thereby removing a coloring material including HMF.

1.3 Monitoring Concentration of HMF

Then, a concentration of HMF included in a filtered fermentation broth, from which the coloring material has been removed, was monitored by using an HPLC method.

Particularly, the concentration of HMF was monitored by injecting 1 mL of the filtered fermentation broth, from which the coloring material has been removed, into an HPLC column (Waters HPLC, Nova-Pak C-18 column, mobile phase: 10% methanol (1 mL/minute, 40° C.), retention time (RT): 1.9 min) and analyzing an HMF peak by using a UV detector (Abs: 285 nm) to measure the concentration of HMF. When the concentration of HMF was 8 mg/L or higher, the coloring material including HMF was removed again by the above procedure. Then, the monitoring of the concentration of HMF was repeated. When the concentration of HMF was lower than 8 mg/L, the process proceeded to the next operation.

1.4 Removing Residual Impurity Ions

15 L of fermentation broth, from which the coloring material has been removed, was consecutively passed through a cation exchange resin column and an anion exchange resin column so as to remove residual impurity ions. The cation exchange resin column and the anion exchange resin column were prepared in the following manner:

Glass columns were arranged by alternately packing cation exchange resins (S2528, available from Lanxess, 500 mL) and anion exchange resins (A4268, available from Lanxess, 100 mL) in the stated order. 2 N HCl and 1N NaOH were used to regenerate functional groups of the exchange resins. Three column volumes (CV) were injected into one of the glass columns. Distilled water was used for the pH at an outlet of the columns to be neutral so that the two columns are set in equilibrium.

1.5 Recovering

15 L of the fermentation broth, from which the residual impurity ions have been removed, was evaporated by using a rotary vacuum evaporator at a temperature of 70° C. and under a pressure of 20 torr. By doing so, a second concentrated fermentation broth from which the residual impurity ions have been removed to have a concentration of 5 volume % of water was obtained. The second concentrated fermentation broth was distilled at a temperature of 140° C. and under a pressure of 5 torr by using thin film distillation, thereby recovering lactic acid or a salt thereof.

### Example 2

Lactic acid and a salt thereof were recovered in the same manner as in Example 1, except that the fermentation broth containing lactic acid was collected after a lapse of about 40 hours instead of about 48 hours from the start of the fermentation in the 30 L fermenter and an activated carbon was not used to remove coloring material including HMF.

### Example 3

Lactic acid and a salt thereof were recovered in the same manner as in Example 1, except that the fermentation broth containing lactic acid was collected after a lapse of about 36 hours instead of about 48 hours from the start of the fermentation in the 30 L fermenter.

### Comparative Example 1

1.1 Preparing Fermentation Broth

The lactic acid-producing yeast strain, Saccharomyces cerevisiae (CEN.PK2-1C), was added to a 30 L fermenter containing a 20 L medium, and fed-batch fermentation was performed thereon, thereby obtaining a fermentation broth containing lactic acid. Medium ingredients included carbon sources, nitrogen sources, trace elements, vitamins, or amino acids, and a specific concentration is as follows: glucose 80 g/L, yeast extract 10 g/L, KH₂PO₄ 2 g/L, (NH₄)₂SO₄ 2 g/L, MgSO₄ 1 g/L, leucine 0.1 g/L, Valine 0.05 g/L, Tryptophan 0.1 g/L, Histidine 0.05 g/L, Uracil 0.1 g/L, Inositol 0.1 g/L, FeSO₄.7H₂O 0.075 g/L, MnSO₄.5H₂O 0.02 g/L, CaCl₂.2H₂O 0.0025 g/L, CuSO₄.5H₂O 0.002 g/L, ZnSO₄.7H₂O 0.02 g/L, H₃BO₃ 0.002 g/L, and Na₂MnO₄ 0.002 g/L. Particularly, the fed-batch fermentation was performed by stirring at 200 rpm by using a single, midlevel, upflow marine-type impeller and by incubating at a temperature of 36° C. Ca(OH)₂ (5M) was automatically added to maintain the pH of the fermentation broth at 3.7.

The fermentation broth containing lactic acid was collected after a lapse of about 48 hours from the start of the fermentation in the 30 L fermenter.

The yeast strain was removed from the fermentation broth containing lactic acid by centrifuging the fermentation broth containing lactic acid by using a centrifuge (Continent512R plus, available from Hanil science industrial, Max RCF: 6,359×g) for 15 minutes. 20 L of a fermentation broth, from which the yeast strain has been removed, was evaporated by using a rotary vacuum evaporator at a temperature of 70° C. and under a pressure of 50 torr for 2 hours. By doing so, ingredients including ethanol were removed therefrom, thereby obtaining a first concentrated fermentation broth, from which the yeast strain has been removed, that is concentrated to 50 volume % of the original volume.

98% sulfuric acid (1 eq.) was added to 10 L of the first concentrated fermentation broth until a pH of the fermentation broth decreased to pH 2.37, thereby causing precipitation of gypsum (Ca(SO₄)₂) and forming lactic acid from calcium lactate. The first concentrated fermentation broth was cooled to room temperature and was vacuum-filtered to obtain a prepared fermentation broth, from which precipitated gypsum (Ca(SO₄)₂) has been removed.

1.2 Removing Residual Impurity Ions

15 L of the prepared fermentation broth was consecutively passed through a cation exchange resin column and an anion exchange resin column so as to remove residual impurity ions. The cation exchange resin column and the anion exchange resin column were prepared in the following manner:

Glass columns were arranged by alternately packing cation exchange resins (S2528, available from Lanxess, 500 mL) and anion exchange resins (A4268, available from Lanxess, 100 mL) in the stated order. Three column volumes (CV) of 2 N HCl and 1N NaOH were used to regenerate functional groups of the exchange resins. After then, resins were rinsed by distilled water in order to neutralize the pH.

1.3 Removing Coloring Material

100 g of activated carbon (C Gran, available from Carbon-norit, 10 w/w lactic acid %) was added to 8 L of the fermentation broth, from which the residual impurity ions have been removed, and the fermentation broth was stirred at room temperature for 2 hours. Here, an average pore diameter of the activated carbon (C Gran) is 3.6 nm, the BET specific surface area is 1200 m²/g, and a pore volume measured by using a BJH method is 0.76 cm³/g. The stirred result was vacuum-filtered by using a disposable filter in order to remove carbon, thereby removing a coloring material.

1.4 Recovering

8 L of fermentation broth, from which the coloring material has been removed, was evaporated by using a rotary vacuum evaporator at a temperature of 70° C. and under a pressure of 20 torr to produce a second concentrated fermentation broth with a final concentration of 5 volume % of water. The second concentrated fermentation broth was distilled at a temperature of 140° C. and under a pressure of 5 torr by using thin film distillation, thereby recovering lactic acid or a salt thereof.

### Comparative Example 2

Lactic acid and a salt thereof were recovered in the same manner as in Comparative Example 1, except that the fermentation broth containing lactic acid was collected after a lapse of about 40 hours instead of about 48 hours from the start of the fermentation in the 30 L fermenter.

### Evaluation Example 1

### Evaluation of HMF-Removing Ability and Lactic Acid Permeability of a Nano-Filter Membrane

The HMF-removing ability and lactic acid permeability was evaluated with the first concentrated fermentation broth prepared as described in Example 1.1 by using a flat sheet-type module employing an NF270 membrane (available from Dow Filmtec, MWCO: 200 Da), a TS40 membrane (available from Trisep, MWCO: 300 Da), and an XN45 membrane (available from Trisep, MWCO: 500 Da), and a membrane surface area of the flat sheet-type module was 0.1 m² at operating pressure (RT) of 40 atmospheric pressure. The HMF-removing ability and lactic acid permeability was evaluated by measuring a residual HMF concentration and a concentration of permeated lactic acid and a salt thereof by using an HPLC method. The result thereof is shown in Table 1 below.

Here, in the 1.1 Preparing fermentation broth of Example 1, a concentration of HMF present in the first concentrated fermentation broth was 100 ppm, and a concentration of the lactic acid and a salt thereof was 144 g/L.

TABLE 1

| | | Concentration of |
|---|---|---|
| | Residual HMF | permeated lactic acid |
| | concentration (ppm) | and salt thereof (g/L) |
| NF270 (MWCO: 200 Da) | 57 | 111 |
| TS40(MWCO: 300 Da) | 60 | 114 |
| XN45(MWCO: 500 Da) | 67 | 121 |
| ESPA4(MWCO: BWRO) | 5 | 12 |

Referring to Table 1, when filtering the first concentrated fermentation broth by using the NF270 membrane, TS40 membrane, and XN45 membrane, the residual HMF concentrations were 57 ppm, 60 ppm, and 67 ppm, respectively. Concentrations of the permeated lactic acid and a salt thereof were 111 g/L, 114 g/L, and 121 g/L, respectively. When filtering the first concentrated fermentation broth by using a conventional ESPA4 membrane, the residual HMF concentration was 5 ppm and the concentration of the penetrated lactic acid and a salt thereof was 12 g/L.

That is, when filtering the first concentrated fermentation broth by using a nano-filter membrane employing the NF270 membrane, TS40 membrane, and XN45 membrane, it was confirmed that the permeability of the lactic acid and a salt thereof was excellent while maintaining the HMF-removing ability, compared to filtering the first concentrated fermentation broth by using a nano-filter membrane employing the conventional ESPA4 membrane.

### Evaluation Example 2

### Physical Properties and HMF-Removing Ability of Activated Carbon Evaluation

Petrodarco, DX, GAC, ROW, or GCN (available from Cabot Norit) activated carbon particles were each added in an amount of 1 wt/wt % to separate 100 mL aliquots of the first concentrated fermentation broth as prepared in Example 1.1 containing 197 ppm of HMF. The first concentrated fermentation broth-activated carbon suspensions were stirred at a temperature of 40° C. for 4 hours then passed through a disposable vacuum-filter.

The HMF-removing ability of the activated carbon pre-adsorption technique was evaluated by measuring a residual HMF concentration after adsorption and vacuum filtration by using an HPLC method. Physical properties of the Petrodarco, DX, GAO, ROW, and GCN (available from Carbon-Norit) activated carbon particles were measured by performing an N₂ absorption-desorption experiment by using a Bell SorpMax (available from Bell). About 0.5 g of samples of the activated carbon were pre-treated at a temperature of 150° C. overnight to remove water, and the samples were maintained under nitrogenic atmosphere by using liquid nitrogen (−77 K). The physical properties of the activated carbon were measured by using a partial pressure equilibrium of stepwise N₂ absorption-desorption to a pressure of 1 bar. From the measured result, a specific surface area and an average pore diameter were calculated by using a BET method, and a pore volume of the activated carbon was calculated by using a BJH method. The result thereof is shown in Table 2 below.

TABLE 2

| | | | | Pore volume |
|---|---|---|---|---|
| | Residual | | | measured by |
| | HMF | BET specific | Average pore | using a BJH |
| | concentration | surface area | diameter | method |
| | (ppm) | (m²/g) | (nm) | (cm³/g) |
| Petrodarco | 121 | 597 | 4.4 | 0.52 |
| DX | 133 | 1045 | 2.9 | 0.45 |
| GAC | 139 | 913 | 2.3 | 0.20 |
| ROW | 157 | 1271 | 2.1 | 0.19 |
| GCN | 174 | 1085 | 1.7 | 0.04 |

As shown in Table 2, the Petrodarco, DX, and GAO particles were most efficient at removing HMF (121 ppm, 133 ppm, and 139 ppm residual HMF, respectively), compared to the ROW and GCN particles (157 ppm and 174 ppm residual HMF, respectively).

Here, when considering the relationship between the HMF-removing ability and the physical properties of the activated carbon, the average pore diameter and pore volume measured by using a BJH method of Petrodarco, DX, and GAO are each 2.3 nm or more and about 0.20 cm³/g to about 0.52 cm³/g. It is confirmed that, compared to the average pore diameter and pore volume measured by using a BJH method of ROW and GCN, the average pore diameter and pore volume measured by using a BJH method of Petrodarco, DX, and GAO are large and have a mesoporous pore volume.

### Evaluation Example 3

### Residual HMF Concentration and Chromaticity of Recovered Lactic Acid Evaluation

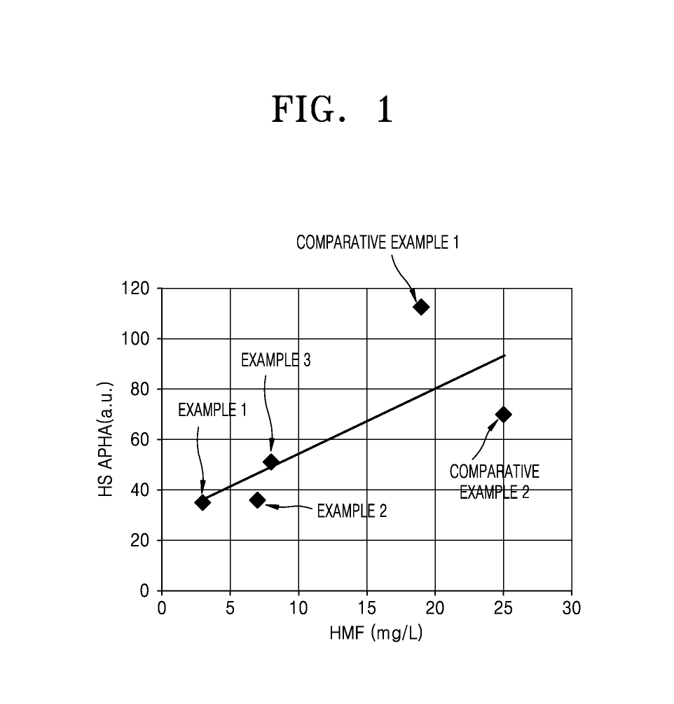

A residual HMF concentration was measured during the 1.3 Monitoring the concentration of HMF in Examples 1 to 3. In Comparative Examples 1 and 2, a residual HMF concentration was measured by using an HPLC method right before the 1.4 Recovering. An HPLC method was also used for monitoring the concentration of HMF in Examples 1 to 3. Also, the recovered lactic acid prepared in Examples 1 to 3 and Comparative Examples 1 and 2 were heated to maintain a temperature of 200° C. for 2 hours, when a concentration of the lactic acid is 90 wt %, to evaluate the chromaticity of the recovered lactic acid by using APHA color (Hazen scale). The result thereof is shown in Table 3 below and FIG. 1.

TABLE 3

| | Residual HMF concentration | |
|---|---|---|
| | (mg/L) | APHA color |
| Example 1 | 3 | 35 |
| Example 2 | 7 | 36 |
| Example 3 | 8 | 51 |
| Comparative | 19 | 113 |
| Example 1 | | |
| Comparative | 25 | 70 |
| Example 2 | | |

As shown in Table 3 and FIG. 1, residual HMF concentrations monitored by HPLC in the filtered fermentation broth in Examples 1 to 3 were 8 mg/L or less. The chromaticities of the recovered lactic acid were APHA color 51 or less when heated to maintain a temperature of 200° C. for 2 hours.

In addition, residual HMF concentrations measured in the 1.3 Monitoring the concentration of HMF of Examples 1 to 3 were lower than residual HMF concentrations measured in the 1.4 Recovering of Comparative Examples 1 and 2. The chromaticities of the lactic acid recovered prepared in Examples 1 to 3 were lower than the chromaticities of the lactic acid recovered according to Comparative Examples 1 and 2.

Accordingly, it is confirmed that the method of separating lactic acid or a salt thereof prepared in Examples 1 to 3 enables obtaining optically transparent high-purity lactic acid or a salt thereof even at a high temperature.

As described above, according to the one or more of the above exemplary embodiments, separating optically transparent high-purity lactic acid or a salt thereof may be achieved by removing a coloring material including HMF.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of separating a lactic acid or a salt thereof, the method comprising:
removing a coloring material comprising hydroxymethylfurfural (HMF) from a fermentation broth, wherein the fermentation broth comprises lactic acid or a salt thereof; and
recovering the lactic acid or a salt thereof from the fermentation broth after the coloring material has been removed.

2. The method of claim 1, wherein removing of the coloring material comprises filtering the fermentation broth.

3. The method of claim 2, wherein filtering of the fermentation broth comprises passing the fermentation broth through a nano-filtration membrane.

4. The method of claim 3, wherein the nano-filtration membrane has a molecular weight cutoff (MWCO) of about 100 Daltons to about 800 Daltons.

5. The method of claim 2, wherein filtering the fermentation broth comprises passing the fermentation broth through a filter after adding a material that adsorbs the coloring material to the fermentation broth so as to batch-adsorb the coloring material.

6. The method of claim 5, wherein the material that adsorbs the coloring material comprises activated carbon, a styrene polymer, a cross-linked polystyrene co-polymer, or combination thereof.

7. The method of claim 6, wherein the material that adsorbs the coloring material comprises activated carbon with an average pore diameter of about 2.3 nm to about 3.5 nm as measured by Brunauer-Emmett-Teller (BET) analysis.

8. The method of claim 6, wherein the material that adsorbs the coloring material comprises activated carbon with a pore volume measured by using a Barett-Joyner-Halenda (BJH) method of about 0.20 cm³/g to about 0.60 cm³/g.

9. The method of claim 1, wherein the coloring material comprises a divalent inorganic ion and/or an organic material comprising HMF.

10. The method of claim 1 further comprising monitoring a concentration of HMF in the fermentation broth after removing the coloring material.

11. The method of claim 10, further comprising maintaining a concentration of HMF in the fermentation broth of less than 8 mg/L.

12. The method of claim 10, further comprising determining that additional HMF removal needs to be performed when the concentration of HMF is 8 mg/L or higher after removing the coloring material.

13. The method of claim 12, further comprising additional HMF removal.

14. The method of claim 10, wherein the concentration of HMF is monitored using a liquid chromatography (LC) method or a high performance liquid chromatography (HPLC) method.

15. The method of claim 1 further comprising removing residual impurity ions before recovering of the lactic acid or a salt thereof.

16. The method of claim 15, wherein removing residual impurity ions comprises passing the fermentation broth, from which the coloring material has been removed, through a nano-filtration membrane.

17. The method of claim 16, wherein removing the residual impurity ions further comprises passing the fermentation broth, from which the coloring material has been removed, through an ion exchange resin or an activated carbon.

18. The method of claim 17 wherein passing the fermentation broth through an ion exchange resin comprises sequentially contacting the fermentation broth with a cation exchange resin and an anion exchange resin.

19. The method of claim 1, wherein recovering the lactic acid or a salt thereof comprises distilling the fermentation broth from which the coloring material has been removed.

20. The method of claim 1, wherein the recovered lactic acid has a chromaticity of about APHA color 50 or less after heating the lactic acid to about 200° C. for about 2 hours at a concentration of 90 wt. %.
